(19) United States
(12) Patent Application Publication
(10) Pub. No.: US 2018/0064714 A1
(43) Pub. Date: Mar. 8, 2018

(54) Process for the Preparation of Amorphous Idelalisib and its Premix

(71) Applicant: Mylan Laboratories Ltd, Hyderabad (IN)

(72) Inventors: Vinayak GORE, Hyderabad (IN); Vinay Kumar SHUKLA, Hyderabad (IN); Dattatraya KANKRALE, Hyderabad (IN); Shardul BHARATI, Hyderabad (IN); Murali BODUPALLI, Hyderabad (IN)

(21) Appl. No.: 15/563,840

(22) PCT Filed: Mar. 12, 2016
(86) PCT No.: PCT/IN2016/050086

§ 371 Date: Oct. 2, 2017

(30) Foreign Application Priority Data
Mar. 13, 2015 (IN) 1268/CHE/2015

(51) Int. Cl. A61K 31/52; A61K 31/517; A61K 47/38; A61K 47/10; B01D 9/00; C07D 473/34; C07D 239/72

(52) U.S. Cl. CPC A61K 31/52; A61K 31/517; A61K 47/38; C07D 239/72; B01D 9/0054; C07D 473/34; A61K 47/10

(57) ABSTRACT

Processes for the preparation of amorphous idelalisib are provided. Processes for the preparation of a premix of amorphous idelalisib are also provided.

### CROSS-REFERENCE TO RELATED APPLICATIONS

This application claims the benefit of Indian provisional patent application no. 1268/CHE/2015 filed on Mar. 13, 2015, which is hereby incorporated by reference in its entirety.

### BACKGROUND OF THE INVENTION

### Field of the Invention

The present invention relates generally to the pharmaceutical arts, and more specifically to amorphous idelalisib and processes for the preparation thereof. The present invention further relates to premixes of amorphous idelalisib and processes for the preparation thereof.

### Background of the Invention

Idelalisib, chemically known as 5-fluoro-3-phenyl-2-[(1S)-1-(7H-purin-6-ylamino)propyl]-4(3H)-quinazolinone, is represented by Formula I below.

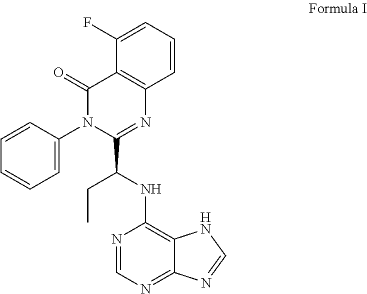 [SMILES: CC[C@H](Nc1ncnc2nc[nH]c12)c1nc2cccc(F)c2c(=O)n1-c1ccccc1]

Idelalisib is marketed under the tradename ZYDELIG® by Gilead Sciences, Inc. ZYDELIG® is indicated for the treatment of patients with chronic lymphocytic leukemia, in combination with rituximab, in patients for whom rituximab alone would be considered appropriate therapy due to other co-morbidities. It is also indicated for treating patients with relapsed follicular B-cell non-Hodgkin lymphoma in patients who have received at least two prior systemic therapies and patients with relapsed small lymphocytic lymphoma in patients who have received at least two prior systemic therapies.

Idelalisib is disclosed in U.S. Pat. No. RE44638. This patent also discloses an ethanol solvate of idelalisib.

International Publication No. WO2013134288 discloses crystalline Forms-I, -II, -III, -IV, -V, -VI and -VII of idelalisib.

International Publication No. WO2015014315 discloses Form-IX of idelalisib, which is a 0.7 hydrate form, as well as Forms-II, -III, -IV, -VIII.

International Publication No. WO2015092810 discloses the amorphous form of idelalisib, process for the preparation thereof, solid dispersions of idelalisib, as well as process for the preparation thereof.

Active pharmaceutical ingredients (APIs) with poor water solubility are often troublesome to incorporate into oral dosage forms having desirable pharmacokinetic properties. Often, solubility, dissolution, and bioavailability of hydrophobic APIs are less than that of more hydrophilic APIs. Thus, it is a general goal of pharmaceutical formulators to develop strategies for improving the pharmacokinetic properties of formulations of APIs with low aqueous solubility, for example, idelalisib.

Thus, the present invention provides a premix of amorphous idelalisib together with one or more pharmaceutically acceptable excipients. The excipients, in some embodiments, may act as carriers for idelalisib, which may impact the overall pharmacokinetic properties of a dosage form into which it may be incorporated. In some embodiments, the excipient is microcrystalline cellulose.

### SUMMARY OF THE INVENTION

One aspect of the present invention provides a method of producing amorphous idelalisib, which may be carried out by the following steps:
a) dissolving idelalisib in a solvent;
b) adding an anti-solvent; and
c) isolating amorphous idelalisib.

Within the context of this method, the solvent may be selected from the group consisting of methanol, ethanol, n-propanol, isopropanol, n-butanol, t-butanol, formic acid, dimethyl sulfoxide, dimethylformamide, dimethylacetamide, 2-methyl tetrahydrofuran, N-methyl-2-pyrrolidone, and mixtures thereof. The anti-solvent may be selected from the group consisting of water, dichloromethane, and mixtures thereof. The addition of the anti-solvent may result in the formation of a precipitate. The isolating step of this method may be achieved by filtering and drying the precipitate, distillation, spray drying, lyophilization, or agitated thin film drying.

Another aspect of the present invention provides a process for the preparation of a premix of amorphous idelalisib,

In one embodiment, a premix of amorphous idelalisib may be prepared out by the following steps:
a) dissolving idelalisib in an organic solvent;
b) adding a pharmaceutically acceptable excipient; and
c) removing the solvent to isolate a premix of amorphous idelalisib.

Within the context of this embodiment, the organic solvent may be an alcohol, for example, methanol, ethanol, isopropanol, n-butanol, and mixtures thereof.

Within the context of this embodiment, the pharmaceutically acceptable excipient may be a polyol, a polymer, a polymer of a polyol, or mixtures thereof. Examples of suitable polyols include mannitol, maltitol, sorbitol, xylitol, erythritol, and isomalt, and mixtures thereof. Examples of suitable polymers include polyethylene glycol (PEG), copolymers of polyethylene glycol and polypropylene glycol (e.g., the families of block copolymers based on ethylene oxide and propylene oxide sold under the PLURONIC® tradename), pentaerythritol, pentaerythritoltetraacetate, polymers of N-vinylpyrrolidone, polyoxyethylene stearates, poly-ε-caprolactone, hypromellose, microcrystalline cellulose, and mixtures thereof.

Within the context of this embodiment, the removal of the solvent to isolate a premix of amorphous idelalisib may be carried out by evaporation, solution concentration, filtration, spray drying, or lyophilization.

In another embodiment, a premix of amorphous idelalisib may be prepared out by the following steps:
a) dissolving idelalisib in a first solvent to form a first solution;
b) adding the solution to a second solvent to form a second solution;
c) adding a pharmaceutically acceptable excipient to the second solution; and
d) isolating a premix of amorphous idelalisib.

Within the context of this embodiment, the first solvent may be, for example, N,N-dimethylformamide, N,N-dimethylacetamide, dimethyl sulfoxide, and mixtures thereof. Optionally, idelalisib may be dissolved in the first solvent at an elevated temperature.

Within the context of the present invention, the second solvent is a solvent in which idelalisib is insoluble or poorly soluble. For example, in some embodiments, the second solvent is water. Within the context of this embodiment, pharmaceutically acceptable excipient may be a polyol, a polymer, or mixtures thereof. Examples of suitable polyols include mannitol, maltitol, sorbitol, xylitol, erythritol, and isomalt, and mixtures thereof. Examples of suitable polymers include polyethylene glycol (PEG), copolymers of polyethylene glycol and polypropylene glycol (e.g., the families of block copolymers based on ethylene oxide and propylene oxide sold under the PLURONIC® tradename), pentaerythritol, pentaerythritoltetraacetate, polymers of N-vinylpyrrolidone, polyoxyethylene stearates, poly-ε-caprolactone, hypromellose, microcrystalline cellulose, and mixtures thereof. In some embodiments, microcrystalline cellulose is used as the pharmaceutically acceptable excipient.

Another aspect of the present invention provides a premix of amorphous idelalisib together with one or more pharmaceutically acceptable excipients. In some embodiments, the premix contains amorphous idelalisib and microcrystalline cellulose. In particular embodiments, microcrystalline cellulose is included in the premix at about 10% w/w to about 50% with respect to total premix mass, for example, at 10% w/w, 30% w/w, or 50% w/w.

Within the context of the present invention, a premix of amorphous idelalisib may be included in a dosage form, for example, an oral dosage form such as a table tor a capsule.

### BRIEF DESCRIPTION OF THE FIGURES

Further aspects of the present disclosure together with additional features contributing thereto and advantages accruing there from will be apparent from the following description of embodiments of the disclosure which are shown in the accompanying drawing figures wherein:

FIG. 1 is a powder x-ray diffraction (PXRD) pattern of amorphous idelalisib; and

FIG. 2 is a PXRD pattern of a premix of amorphous idelalisib.

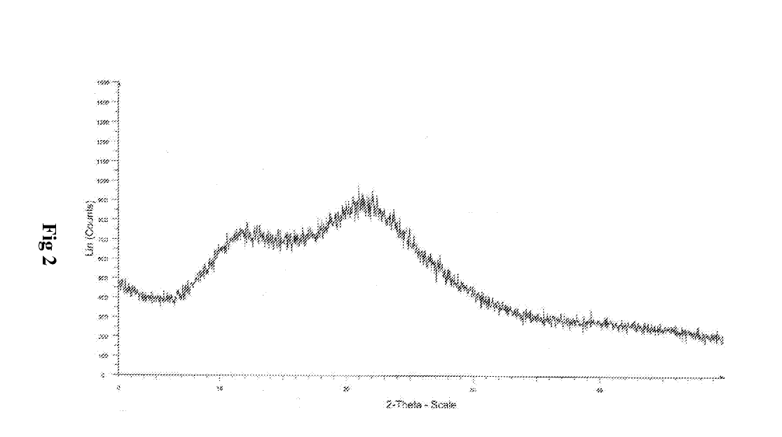

### DETAILED DESCRIPTION OF THE INVENTION

It is to be understood that the description of the present invention has been simplified to illustrate elements that are relevant for a clear understanding of the invention.

The present invention encompasses stable premixes of amorphous idelalisib useful in generating pharmaceutical formulations for administration to patients in need thereof. Within the context of the present invention, amorphous idelalisib may be generated according to the following processes.

Amorphous idelalisib may be prepared by the following steps:
a) dissolving idelalisib in a solvent;
b) adding an anti-solvent; and
c) isolating amorphous idelalisib.

In the first step of this process, idelalisib may be dissolved in a solvent. Within the context of this method, the idelalisib starting material may be in a variety of forms, for example, any polymorphic crystalline form or a solvate. The solvent may be, for example, methanol, ethanol, n-propanol, isopropanol, n-butanol, t-butanol, formic acid, dimethyl sulfoxide, dimethylformamide, dimethylacetamide, 2-methyl tetrahydrofuran, N-methyl-2-pyrrolidone, or mixtures thereof.

Next, an anti-solvent may be added. The anti-solvent may be, for example, water, dichloromethane, or a mixture thereof. Within the context of the present method, the addition of the anti-solvent may cause a precipitate to form.

According to this method, amorphous idelalisib may then be isolated. For example, isolation may be carried out by filtering the solution and drying the obtained solid, distilling off the solution, spray drying, lyophilization, or agitated thin film drying.

In yet another method, amorphous idelalisib may be prepared by the following steps:
a) dissolving idelalisib in a solvent; and
b) removing the solvent to isolate amorphous idelalisib.

In the first step of this method, idelalisib may be dissolved in a solvent. Within the context of this method, the idelalisib starting material may be in a variety of forms, for example, any polymorphic crystalline form or a solvate. The solvent may be, for example, methanol, ethanol, n-propanol, t-butanol, isopropanol, n-butanol, dimethylformamide, dimethylacetamide, dimethyl sulfoxide, or mixtures thereof. In some implementations of this method, ethanol is used in this step as the solvent.

The solvent may then be removed to isolate amorphous idelalisib. This step may be carried out by conventional techniques well known to one in the art, such as, for example, evaporation, concentration of the solution, distillation, agitated thin film drying, spray drying, or lyophilization. In some implementations of this method, distillation is used to remove the solvent.

The amorphous idelalisib disclosed herein may be characterized by a PXRD pattern. Thus, the PXRD pattern of amorphous idelalisib was measured on BRUKER D-8 Discover powder diffractometer equipped with a goniometer of θ/2θ configuration and Lynx Eyedetector. The Cu-anode X-ray tube was operated at 40 kV and 30 mA. The experiments were conducted over the 2θ range of 2.0°-50.0°, 0.030° step size and 0.4 seconds step time.

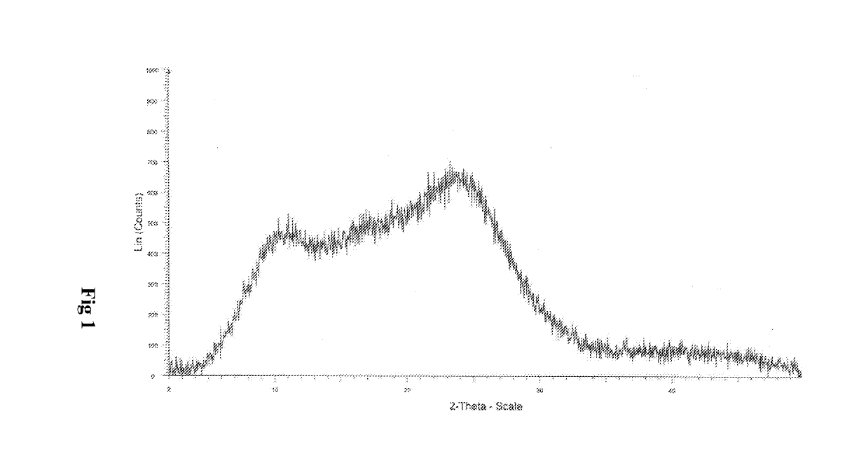

In one embodiment, the amorphous idelalisib of the present invention may be characterized as amorphous by the powder x-ray diffraction (PXRD) pattern in FIG. 1.

One aspect of the present invention provides pharmaceutical premixes of idelalisib and at least one pharmaceutical excipient, and methods of preparation thereof.

As used herein, the term“premix” refers to solid mixtures of idelalisib and at least one pharmaceutical excipient. The premixes of the present invention may provide multiple benefits in preparing formulations of idelalisib, for example, improved processability, increased stability of the pharmaceutical formulation or API, or improved pharmacokinetic properties of the pharmaceutical formulation. It is further recognized that idelalisib, as a hydrophobic drug, commonly has low bioavailability. Association of idelalisib with a hydrophilic excipient may help improve the bioavailability of the API.

One embodiment provides a dispersion of idelalisib together with a pharmaceutically acceptable excipient. Within the context of this embodiment, the pharmaceutically acceptable excipient may be a dispersing agent. In some embodiments, the dispersing agent is hydrophilic and forms a hydrophilic matrix in which idelalisib is dispersed. The dispersing agent and hydrophilic matrix may be crystalline or amorphous. Idelalisib may be dispersed molecularly, in amorphous particles, or in crystalline particles.

Examples of particular embodiments of premixes of amorphous idelalisib of this invention may be characterized as amorphous by PXRD. Thus, the PXRD pattern of several embodiments of premixes of amorphous idelalisib were measured on BRUKER D-8 Discover powder diffractometer equipped with a goniometer of θ/2θ configuration and Lynx Eyedetector. The Cu-anode X-ray tube was operated at 40 kV and 30 mA. The experiments were conducted over the 20 range of 2.0°-50.0°, 0.030° step size and 0.4 seconds step time.

The present invention further provides a process for the preparation of a premix of idelalisib together with one or more pharmaceutically acceptable excipients.

In one embodiment, a premix of amorphous idelalisib may be prepared by the following steps:
a) dissolving idelalisib in an organic solvent;
b) adding one or more pharmaceutically acceptable excipients; and
c) removing the solvent to isolate a premix of amorphous idelalisib.

In the first step of this embodiment, idelalisib may be dissolved in an organic solvent. Within the context of this method, the idelalisib starting material may be in a variety of forms, for example, the amorphous form, any polymorphic crystalline form, or a solvate. The solvent may be, for example, a polar solvent. In some embodiments, an alcohol solvent, for example, methanol, ethanol, isopropanol, n-butanol, or mixtures thereof was found to be useful. In some particularly useful embodiments, methanol is used to dissolve idelalisib.

Next, one or more pharmaceutically acceptable excipients may be added. Within the context of the present embodiment the pharmaceutically acceptable excipient may be a polyol, a polymer (including polymeric polyols), or mixtures thereof. Examples of suitable polyols include mannitol, maltitol, sorbitol, xylitol, erythritol, and isomalt, and mixtures thereof. Examples of suitable polymers include polyethylene glycol (PEG), copolymers of polyethylene glycol and polypropylene glycol (e.g., the families of block copolymers based on ethylene oxide and propylene oxide sold under the PLURONIC® tradename), pentaerythritol and pharmaceutically acceptable derivatives thereof (for example, pentaerythritoltetraacetate), polymers of N-vinylpyrrolidone (e.g., polyvinylpyrrolidone and copolymers of N-vinylpyrrolidone and vinyl acetate), polyoxyethylene stearates, caprolactones (for example, poly-ε-caprolactone), hypromellose, microcrystalline cellulose, and mixtures thereof. Examples suitable polymers of N-vinylpyrrolidone include, for example, polyvinylpyrrolidone and copolymers of N-vinylpyrrolidone and vinyl acetate.

Within the context of this embodiment of the present disclosure, the pharmaceutically acceptable excipient may be added to the idelalisib solution such that the final w/w % of pharmaceutically acceptable excipient to total composition mass is from about 10% w/w to about 50% w/w, which may be about 10% w/w, 15% w/w, 20% w/w, 25% w/w, 30% w/w, 35% w/w, 40% w/w, 45% w/w, 50% w/w, or between any of the aforementioned w/w percentages, including the ranges of about 10%-40%, 10%-30%, 10%-20%, 20%-50%, 20%-40%, 20%-30%, 30%-50%, 30%-40%, and 40%-50% w/w. In some embodiments of the present disclosure, combining microcrystalline cellulose at concentrations recited above, including from about 10% to 50% w/w, with idelalisib may be found to be useful.

The solvent may then be removed, for example, by evaporation, concentration of the solution, filtration, spray drying, or lyophilization. Within the context of this embodiment, removal of the solvent may result in the formation of a premix in which the drug is dispersed throughout the pharmaceutically acceptable excipient.

In some embodiments, removal of the solvent may be optionally carried out at an elevated temperature. For example, in some embodiments, the solvent was removed at a temperature of about 45° C. to about 50° C.

In yet another embodiment, a premix of an amorphous form of idelalisib may be prepared by the following steps:
a) dissolving idelalisib in an organic solvent to form a first solution;
b) adding the solution to a second solvent to form a second solution;
c) adding one or more pharmaceutically acceptable excipient to the second solution; and
d) isolating the premix of amorphous idelalisib.

According to the present embodiment, idelalisib may be dissolved in a solvent to form a first solution. Within the context of this method, the idelalisib starting material may be in a variety of forms, for example, the amorphous form, any polymorphic crystalline form, or a solvate.

Within the context of this embodiment, the solvent may be dimethyl sulfoxide, N,N-dimethylformamide, N,N-dimethylacetamide, or mixtures thereof. Optionally, the first solution may be then heated, for example, to reflux. Within the context of this embodiment, heating may improve dissolution of idelalisib. In some embodiments, however, complete dissolution of the idelalisib may not be necessary before proceeding to the next step in the process.

According to the present embodiment, the first solution may then be added to a second solvent.

Within the context of this embodiment, the idelalisib may be insoluble or poorly soluble in the second solvent and addition of the solvent may cause idelalisib to precipitate out of solution. One of skill in the art will be familiar with a variety of suitable solvents that have characteristics to facilitate this process. For example, in some embodiments, water is used as the second solvent.

Next, a pharmaceutically acceptable excipient may be added to the second solution. Within the context of the present embodiment the pharmaceutically acceptable excipient may be a polyol, a polymer (including polymeric polyols), or mixtures thereof. Examples of suitable polyols include mannitol, maltitol, sorbitol, xylitol, erythritol, and isomalt, and mixtures thereof. Examples of suitable polymers include polyethylene glycol, copolymers of polyethylene glycol and polypropylene glycol, pentaerythritol (and pharmaceutically acceptable derivatives thereof, for example, pentaerythritoltetraacetate), polymers of N-vinylpyrrolidone, polyoxyethylene stearates, caprolactones (for example, poly-ε-caprolactone), hypromellose, microcrystalline cellulose, and mixtures thereof. Examples suitable polymers of N-vinylpyrrolidone include, for example, polyvinylpyrrolidone and copolymers of N-vinylpyrrolidone and vinyl acetate. In some embodiment, the polyvinylpyrrolidone has a K-value of about 30. In certain embodiments, mannitol is used as the pharmaceutical excipient. In other embodiments, microcrystalline cellulose is used as a pharmaceutical excipient.

Within the context of this embodiment of the present disclosure, the pharmaceutically acceptable excipient may be added to the idelalisib solution such that the final w/w % of pharmaceutically acceptable excipient to total composition mass is from about 10% w/w to about 50% w/w, which may be about 10% w/w, 15% w/w, 20% w/w, 25% w/w, 30% w/w, 35% w/w, 40% w/w, 45% w/w, 50% w/w, or between any of the aforementioned w/w percentages, including the ranges of about 10%-40%, 10%-30%, 10%-20%, 20%-50%, 20%-40%, 20%-30%, 30%-50%, 30%-40%, and 40%-50% w/w. In some embodiments of the present disclosure, combining microcrystalline cellulose at concentrations recited above, including from about 10% to 50% w/w, with idelalisib may be found to be useful.

Next, the solid formed may be isolated to obtain a premix of amorphous idelalisib. For example, isolation may be carried out by filtering the solution to produce a solid, which may then be further processed, for example, by drying, to obtain the final amorphous idelalisib product.

The premixes of amorphous idelalisib disclosed herein may be used in generating pharmaceutical dosage forms that are useful in the treatment of patients with chronic lymphocytic leukemia, in combination with rituximab, in patients for whom rituximab alone would be considered appropriate therapy due to other co-morbidities. Pharmaceutical dosage forms containing premixes of amorphous idelalisib may also be used for treating patients with relapsed follicular B-cell non-Hodgkin lymphoma in patients who have received at least two prior systemic therapies and patients with relapsed small lymphocytic lymphoma in patients who have received at least two prior systemic therapies.

The premixes of amorphous idelalisib disclosed herein may be incorporated into oral pharmaceutical dosage forms, for example, a capsule or tablet. The dosage form may include additional excipients, for example, microcrystalline cellulose, hydroxypropyl cellulose, croscarmellose sodium, sodium starch glycolate, colloidal silicon dioxide, magnesium stearate, and mixtures thereof. The dosage form may, in some embodiments, be coated with a shell or film that includes additional excipients, artificial flavorings, artificial colorings, and mixtures thereof. For example, the shell or film may include such excipients as polyethylene glycol, talc, polyvinyl alcohol, titanium dioxide, red iron oxide, and mixtures thereof.

Within the context of the present disclosure, the amorphous form of idelalisib as well as premixes of amorphous idelalisib of the present disclosure may exhibit long-term physical and chemical stability. Within the context of the present invention, the physical and chemical stability of amorphous idelalisib and premixes of amorphous idelalisib disclosed herein may be determined by storing the samples at either 40° C./75% relative humidity (RH), at 25° C./60% relative humidity (RH), or at 5° C.±3° C. for 6 months and analyzing the stored material by PXRD to determine polymorph integrity.

As used herein, a compound or pharmaceutical composition is considered “stable” where the HPLC purity of the compound or premix changes by less than about 1% when stored at 5° C.±3° C. and/or at 25° C./60% relative humidity (RH). In certain embodiments, the “stable” compound or premix is stored at 5° C.±3° C. In other embodiments, the “stable” compound or premix is stored at 25° C./60% relative humidity (RH).

In some embodiments, amorphous idelalisib prepared to methods disclosed herein shows no change in PXRD pattern (i.e., remains amorphous and stable) when stored for six months at 5° C.±3° C., 25° C./60% RH, or at 40° C./75% RH conditions. Likewise, certain embodiments of premixes of amorphous idelalisib, such as those prepared with microcrystalline cellulose, prepared as per the examples disclosed herein, also show no change in PXRD pattern (i.e., remains amorphous) when stored for six months at 5° C.±3° C., 25° C./60% RH, or at 40° C./75% RH conditions.

TABLE 1

| | | Premixes of amorphous idelalisib | | |
|---|---|---|---|---|
| | | 10% w/w | 30% w/w | 50% w/w |
| | | microcrystal- | microcrystal- | microcrystal- |
| | Amorphous | line | line | line |
| Condition | Idelalisib | cellulose | cellulose | cellulose |

at 40° C./75% RH

| | | | | |
|---|---|---|---|---|
| Initial PXRD | Amorphous | Amorphous | Amorphous | Amorphous |
| analysis | | | | |
| 1 month | Stable | Stable | Stable | Stable |
| 3 months | Stable | Stable | Stable | Stable |
| 6 months | Stable | Stable | Stable | Stable |

at 25° C./60% RH

| | | | | |
|---|---|---|---|---|
| Initial PXRD | Amorphous | Amorphous | Amorphous | Amorphous |
| analysis | | | | |
| 1 month | Stable | Stable | Stable | Stable |
| 3 months | Stable | Stable | Stable | Stable |
| 6 months | Stable | Stable | Stable | Stable |

at 5° C. ± 3° C.

| | | | | |
|---|---|---|---|---|
| Initial PXRD | Amorphous | Amorphous | Amorphous | Amorphous |
| analysis | | | | |
| 1 month | Stable | Stable | Stable | Stable |
| 3 months | Stable | Stable | Stable | Stable |
| 6 months | Stable | Stable | Stable | Stable |

Crystalline solids normally require a substantial amount of energy for dissolution due to their highly organized, lattice-like structures. For example, the energy required for a drug molecule to escape from a crystal is more than from an amorphous or a non-crystalline form. (Econno T., Chem. Pharm. Bull., 1990; 38: 2003-2007; which is hereby incorporated by reference in its entirety)). Therefore, incorporating amorphous forms of active pharmaceutical ingredients may be advantageous over crystalline forms, as amorphous forms may exhibit enhanced properties such as increased dissolution rates, increased solubility, and increased bioavailability. However, amorphous forms are sometimes unstable when compared to crystalline solids. In some cases, amorphous solids will begin to crystalize over time if such a transition is thermodynamically favorable.

Therefore, the amorphous idelalisib, prepared by methods disclosed herein and exhibiting the stability profiles as illustrated in Table 1, may provide added pharmaceutical benefits over idelalisib in crystalline form, particularly when incorporating the API into a dosage form.

Idelalisibis poorly soluble in water. APIs with poor water solubility are often troublesome to incorporate into oral dosage forms having desirable pharmacokinetic properties. Often, solubility, dissolution, and bioavailability of hydrophobic APIs are less than that of more hydrophilic APIs. Thus, it is a general goal of pharmaceutical formulators to develop strategies to improve the pharmacokinetic properties of formulations of drugs with low aqueous solubility, for example, idelalisib. Therefore, a premix of amorphous idelalisib, prepared by methods disclosed herein and exhibiting the stability profiles as illustrated in Table 1, may provide added pharmaceutical benefits over inclusion of idelalisib without any pharmaceutical excipients or carriers, particularly when incorporating the idelalisib dispersion into a dosage form.

Within the context of the present invention, dosage forms containing amorphous idelalisib or premixes thereof may have between about 100 mg and 150 mg of idelalisib per dose, including 100 mg and 150 mg of idelalisib.

In view of the above description and the examples below, one of ordinary skill in the art will be able to practice the invention as claimed without undue experimentation. The foregoing will be better understood with reference to the following examples that detail certain procedures for the preparation of molecules according to the present invention. All references made to these examples are for the purposes of illustration. The following examples should not be considered exhaustive, but merely illustrative of only a few of the many aspects and embodiments contemplated by the present disclosure.

### EXAMPLES

### Example 1

### Preparation of 5-fluoro-2H-3,1-benzoxazine-2,4-dione

2-amino-6-fluoro-benzoic acid (50 g, 0.32 mole) was dissolved in acetonitrile (500 mL). Pyridine (51 g, 0.64 moles) was added. A solution of triphosgene (47.8 g (0.18 mole) in dichloromethane) was slowly added while maintaining the reaction mixture temperature between 50 to 60° C. The reaction was maintained at same temperature for next 4 to 6 hours. After completion of the reaction, solvent was removed by vacuum distillation. To the residue, 500 mL of water was added and stirred to get a slurry. The slurry was filtered, washed with water, and dried to get 5-fluoro-2H-3,1-benzoxazine-2,4-dione as off-white solid (52 g, 89% molar).

### Example 2

### Preparation of 2-amino-6-fluoro-N-phenyl-benzamide

5-fluoro-2H-3,1-benzoxazine-2,4-dione (50 g, 0.27 mole) was dissolved in acetonitrile (500 mL) and the mixture was heated to 40° C. Aniline (25.7 g, 0.27 mole) was added and the reaction mixture was heated to 80° C.±82° C. and maintained at this temperature for 5 to 6 hours. After completion of the reaction, the solvent was removed by vacuum distillation and the residue was dissolved in methanol (200 mL). The resulting solution was quenched with water (1000 mL). The isolated solid was filtered, the solid was washed with water, and then sucked dry. Finally, solid was dried at 50° C.-60° C. for 6 to 8 hours to give 2-amino-6-fluoro-N-phenyl-benzamide brownish to off-white solid (42 g, 82.5%).

### Example 3

### Preparation of (S)-tert-butyl-1-(3-fluoro-2-(phenylcarbamoyl)phenylamino)-1-oxobutan-2-yl carbamate

N-methyl morpholine (48.22 g, 0.48 mole) was added to a solution of (S)-2-(tert-butoxycarbonylamino)butanoic acid (88.2 g, 0.43 mole) in tetrahydrofuran (100 mL). The resulting mixture was cooled to 0° C.-−5° C. and a solution of isobutyl chloroformate (59.3 g (0.43 mole) in 100 mL of tetrahydrofuran) was added. A solution of 2-amino-6-fluoro-N-phenyl-benzamide (50 g, 0.27 mole) in tetrahydrofuran (200 mL) was slowly added at 0° C.-−5° C. The reaction mixture temperature was raised to 25° C.-28° C. and the solution was stirred for 20 hours. After completion of the reaction, water (500 mL) and ethyl acetate (500 mL) were added to the reaction mixture. The organic layer was separated, washed with water, and dried on sodium sulfate. Solvent was removed by vacuum distillation. The residue was dissolved in tetrahydrofuran (250 mL) and n-heptane was added to precipitate (5)-tert-butyl-1-(3-fluoro-2-(phenyl carbamoyl)phenylamino)-1-oxobutan-2-yl carbamateas a solid. The solution was filtered, the solid was washed with n-heptane, and finally dried at 55° C. under vacuum to get a light brown colored solid (82 g, 90% molar).

### Example 4

### Preparation of (S)-tert-butyl-[1-(5-fluoro-4-oxo-3-phenyl-3,4-dihydroquinazolin-2-yl)propyl]-carbamate

(S)-tert-butyl-1-(3-fluoro-2-(phenyl carbamoyl) phenyl amino)-1-oxobutan-2-yl carbamate (50 g, 0.12 mole) was dissolved in acetonitrile (1500 mL). Triethylamine (577.4 g, 47.5 mole) was added and the mixture was cooled to 15 to 20° C. Trimethylsilyl chloride (196.1 g, 15 mole) was then slowly added. The reaction mixture was transferred to a pressure reactor and stirred at 90° C.-95° C. for 24 hours at pressure (˜1 to 2 kg/cm²). After completion of the reaction, the reaction mixture was concentrated by vacuum distillation. The residue was cooled and 15% w/v solution of sodium bicarbonate was added and the reaction mixture was extracted with ethyl acetate (2×250 mL). The organic layer was washed with water and dried on sodium sulfate. Finally the solution was concentrated by distillation under vacuum to get (S)-tert-butyl [1-(5-fluoro-4-oxo-3-phenyl-3,4-dihydroquinazolin-2-yl) propyl]-carbamate as brown color solid (45 g, 94% molar yield).

### Example 5

### Preparation of 2-(1-aminopropyl)-5-fluoro-3-phenyl-3H-quinazolin-4-one

(S)-tert-butyl-[1-(5-fluoro-4-oxo-3-phenyl-3,4-dihydroquinazolin-2-yl)propyl] -carbamate (50 g, 0.12 mole) was dissolved in dichloromethane (100 mL) and cooled to 8° C.-10° C. Trifluoroacetic acid (150 g, 1.31 mole) was slowly added. The reaction mixture was stirred at 25° C.-28° C. for 3 to 6 hours. After completion of reaction, the reaction mixture was concentrated and the residue was dissolved in dichloromethane and water was added. The pH was adjusted to ˜10 with a 10% sodium carbonate solution. The organic layer was separated, washed with water, dried on sodium sulfate, and then concentrated under vacuum. The residue was stirred with Diisopropyl ether (DIPE) to get a solid, which was filtered and washed with DIPE and dried at 55° C.-60° C. under vacuum to get 2-(1-aminopropyl)-5-fluoro-3-phenyl-3H-quinazolin-4-one (33.2 g, 88% molar).

### Example 6

### Preparation of 5-fluoro-3-phenyl-2-[(1S)-1-(7H-purin-6-ylamino)propyl]-4(3H)-quinazolinone [Idelalisib]

A mixture of 2-(1-aminopropyl)-5-fluoro-3-phenyl-3H-quinazolin-4-one (50 g, 0.17 mole) and 6-bromopurine (37.5 g, 0.18 mole) was taken in tert-butanol (500 mL). To this solution, 43.4 g DIPEA [N,N,-diisopropylethylamine] was added and the reaction mixture was stirred at 85° C.-90° C. for 20 to 25 hours. After completion of the reaction, the reaction mixture was concentrated by distillation under vacuum and the residue was dissolved in methanol (500 mL). This solution was slowly added to water (5000 mL) and stirred for next 40 to 60 minutes. The solid was filtered then sucked dried. Finally, the solid was dried at 50° C.-55° C. under vacuum for 4 to 5 hours to get 5-fluoro-3-phenyl-2[(1S)-1-(7H-purin-6-ylamino)propyl]-4(3H)-quinazolinone (idelalisib) (62.5 g, 89% molar).

### Example 7

### Preparation of Amorphous Idelalisib

5-fluoro-3-phenyl-2-[1S)-1-(7H-purin-6-ylamino)propyl]-4(3H)-quinazolinone (5 g) was dissolved in absolute ethanol (250 mL) at 60° C. The solution was filtered through a celite bed and the clear solution was distilled under reduced pressure to remove solvent. After complete removal of solvent, a powdery solid was obtained. A PXRD analysis identified the solid as an amorphous form of idelalisib (4.5 g).

### Example 8

### Preparation of a Premix of Amorphous Idelalisib with 50% PVP K-30

A mixture of idelalisib (1.0 g) and polyvinylpyrrolidone (PVP K-30, 1.0 g) was dissolved in methanol (80 mL) and stirred at 25° C.-30° C. to get a clear solution. This solution was concentrated at 45° C.-50° C. under vacuum. After complete removal of the solvent, a residue was obtained as a fluffy solid [idelalisib dispersion] (2.0 g).

### Example 9

### Preparation of Amorphous Idelalisib

(S)-2-(1-(9H-purin-6-ylamino)propyl)-5-fluoro-3-phenylquinazolin-4(3H)-one semisolid mass (2 g) was dissolved in n-propanol (20 mL) at 50° C. The solution was allowed to cool to 25° C. and poured into 200 mL demineralized water precooled to 10° C.-15° C. The resultant mixture was stirred at 10° C.-15° C. for 30 minutes then filtered by Buchner funnel to isolate a solid. The solid was washed with water, suck dried, then dried in a vacuum oven at 50° C.-55° C. for 1 hour. An obtained PXRD pattern indicated that the solid was an amorphous form of idelalisib (1.8 g).

### Example 10

### Preparation of Amorphous Idelalisib

(S)-2-(1-(9H-purin-6-ylamino)propyl)-5-fluoro-3-phenylquinazolin-4(3H)-one semisolid mass (2 g) was dissolved in isopropanol (20 mL) at 50° C. The solution was allowed to cool to 25° C. and poured into 200 mL demineralized water precooled to 10° C.-15° C. The resultant mixture was stirred at 10° C.-15° C. for 30 minutes, filtered by Buchner funnel, and the isolated solid was washed with water then suck dried. The solid was then dried in a vacuum oven at 50° C.-55° C. for 1 hour. An obtained PXRD pattern indicated that the solid was an amorphous form of idelalisib (1.8 g).

### Example 11

### Preparation of Amorphous Idelalisib

(S)-2-(1-(9H-purin-6-ylamino)propyl)-5-fluoro-3-phenylquinazolin-4(3H)-one (2 g) was dissolved in tert-butanol (20 mL) at 60° C. The clear solution was distilled at 50° C.-55° C. under reduced pressure to remove the solvent. n-heptane was then added (to facilitate removal of tert-butanol) and the solvent removed under vacuum to yield a powdery solid. An obtained PXRD pattern indicated that the solid was an amorphous form of idelalisib (1.9 g).

### Example 12

### Preparation of Amorphous Idelalisib

(S)-2-(1-(9H-purin-6-ylamino)propyl)-5-fluoro-3-phenylquinazolin-4(3H)-one semisolid mass (2 g) was dissolved in dimethylformamide (DMF, 20 mL) at 50° C. The solution was allowed to cool to 25° C. and poured into 200 mL demineralized water precooled to 10° C.-5° C. The resultant mixture was stirred at 10° C.-15° C. for 30 minutes then filtered by Buchner funnel The obtained solid was washed with water, suck dried, then dried in a vacuum oven at 50° C.-55° C. for 1 hour. An obtained PXRD pattern indicated that the solid was an amorphous form of idelalisib (1.6 g).

### Example 13

### Preparation of Amorphous Idelalisib

(S)-2-(1-(9H-purin-6-ylamino)propyl)-5-fluoro-3-phenylquinazolin-4(3H)-one semisolid mass (2 g) was dissolved in dimethylacetamide (DMAc, 20 mL) at 50° C. The solution was allowed to cool at 25° C. and poured into 200 mL demineralized water precooled to 10° C.-15° C. The resultant mixture was stirred at 10° C.-15° C. for 30 minutes, then filtered by Buchner funnel. The solid obtained was washed with water, suck dried, then dried in a vacuum oven at 50° C.-55° C. for 1 hour. An obtained PXRD pattern indicated that the solid was an amorphous form of idelalisib (1.7 g).

### Example 14

### Preparation of Amorphous Idelalisib

(S)-2-(1-(9H-purin-6-ylamino)propyl)-5-fluoro-3-phenylquinazolin-4(3H)-one semisolid mass (2 g) was dissolved in dimethyl sulfoxide (DMSO, 10 mL) at 50° C. The solution was allowed to cool to 25 ° C. and poured into 100 mL demineralized water precooled to 10 ° C.-15° C. The resultant mixture stirred at 10° C-15° C. for 30 minute, then filtered by Buchner funnel. The obtained solid was washed with water, suck dried, then dried in a vacuum oven at 50° C.-55° C. for 1 hour. An obtained PXRD pattern indicated that the solid was an amorphous form of idelalisib (1.9 g).

### Example 15

### Preparation of Amorphous Idelalisib

Idelalisib (5 g) and dimethyl sulfoxide (10 mL) were added into a round bottom flask at 25° C.-30° C. The reaction mass was stirred at 25° C.-30° C. for 20-30 minutes to get a clear solution. Purified water (150 mL) was added at 25° C.-30° C. over 30 -60 minutes and stirred at the same temperature for 60 minutes. The solution was filtered, and the solid obtained was washed with water (20 mL) and dried at 25° C.-30° C. under vacuum for 16 hours. The resulting product was identified as amorphous idelalisib (4.7 g).

### Example 16

### Preparation of Amorphous Idelalisib

Idelalisib (200 mg) was dissolved in formic acid (0.5 mL) at 50° C. then cooled to 25° C.-30° C. Water (5 mL) was added and the solution was stirred at the same temperature for 20 hours. The solution was filtered and the solid obtained was dried at 35° C. under vacuum for 2 hours. The resulting product was identified as amorphous form of idelalisib (180 mg). Whether this is

### Example 17

### Preparation of Amorphous Idelalisib

Idelalisib (200 mg) was dissolved in 2-methyl tetrahydrofuran (10 mL) at 60° C. The clear solution was then cooled to 25° C.-30° C. The solvent was slowly evaporated off at same temperature for 24 hours. No precipitate was observed. Water (20 mL) was then added, the precipitate obtained was filtered out of the solution, and identified as an amorphous form of idelalisib.

### Example 18

### Preparation of Amorphous Idelalisib

Idelalisib (200 mg) was dissolved in N-methyl-2-pyrrolidone (1 mL) at 80° C. The clear solution was then cooled to 25° C.-30° C. The solvent was slowly evaporated off at same temperature for 24 hours. No precipitate was observed. Water (10 mL) was then added and the precipitate obtained was filtered out and identified as an amorphous form of idelalisib.

### Example 19

### Preparation of a Premix of Amorphous Idelalisib with 10% w/w Microcrystalline Cellulose

Idelalisib (5 g) was dissolved in dimethyl sulfoxide (12.5 mL) at 25° C.-30° C. The solution was filtered to remove undissolved particulate and washed with dimethyl sulfoxide (2.5 mL) The clear solution of idelalisib was added into water (150 mL) maintained at 25° C.-30° C. over 30 minutes. The reaction mass was further stirred for 30 minutes. Microcrystalline cellulose (0.556 g, Grade: AVICEL PH 101) was then added into the reaction mass and which was then stirred for 1 hour. The solid obtained was filtered out, washed with water (20 mL), and dried at 30° C. under vacuum for 16 hours. It was then further dried at 40° C. under vacuum for 24 hours. The resulting product obtained was identified by PXRD as a premix of amorphous idelalisib (5 g).

### Example 20

### Preparation of a Premix of Amorphous Idelalisib with 30% w/w Microcrystalline Cellulose

Idelalisib (5 g) was dissolved in dimethyl sulfoxide (12.5 mL) at 25° C.-30° C. The solution was filtered to remove undissolved particulate and washed with dimethyl sulfoxide (2.5 mL) The clear solution of idelalisib was added into water (150 mL) maintained at 25° C.-30° C. over 30 minutes. The reaction mass was further stirred for 30 minutes. Microcrystalline cellulose (2.142 g, Grade: AVICEL PH 101) was then added into the reaction mass and stirred for 1 hour. The solid obtained was filtered, washed with water (20 mL), and dried at 30° C. under vacuum for 16 hours. It was then further dried at 40° C. under vacuum for 24 hours. The resulting product obtained was identified by PXRD as a premix of amorphous idelalisib (6.8 g).

### Example 22

### Preparation of a Premix of Amorphous Idelalisib with 50% w/w Microcrystalline Cellulose

Idelalisib (5 g) was dissolved in dimethyl sulfoxide (12.5 mL) at 25° C.-30° C. The solution was filtered to remove undissolved particulate and washed with dimethyl sulfoxide (2.5 mL). The clear solution of idelalisib was added into water (150 mL) maintained at 25° C.-30° C. over 30 minutes. The reaction mass was further stirred for 30 minutes. Microcrystalline cellulose (5 g, Grade: AVICEL PH 101) was then added into the reaction mass and stirred for 1 hour. The solid obtained was filtered, washed with water (20 mL), and dried at 30° C. under vacuum for 16 hours. It was then further dried at 40° C. under vacuum for 24 hours. The resulting product obtained was identified by PXRD as a premix of amorphous idelalisib (9.8 g).

We claim:

1. A method of producing amorphous idelalisib, comprising the steps of:
a) dissolving idelalisib in a solvent;
b) adding an anti-solvent; and
c) isolating amorphous idelalisib.

2. The method of claim 1, wherein the solvent is selected from the group consisting of methanol, ethanol, n-propanol, isopropanol, n-butanol, t-butanol, formic acid, dimethyl sulfoxide, dimethylformamide, dimethylacetamide, 2-methyl tetrahydrofuran, N-methyl-2-pyrrolidone, and mixtures thereof.

3. The method of claim 1, wherein the anti-solvent is selected from the group consisting of water, dichloromethane, and mixtures thereof.

4. The method of claim 1, wherein the adding of the anti-solvent results in the formation of a precipitate.

5. The method of claim 1, wherein the isolating step is achieved by filtering and drying the precipitate, distillation, spray drying, lyophilization, or agitated thin film drying.

6. A process for the preparation of a premix of amorphous idelalisib, comprising the steps of:
a) dissolving idelalisib in an organic solvent;
b) adding a pharmaceutically acceptable excipient; and
c) removing the solvent to isolate a premix of amorphous idelalisib.

7. The process according to claim 6, wherein the organic solvent is an alcohol.

8. The process according to claim 7, wherein the alcohol is selected from the group consisting of methanol, ethanol, isopropanol, n-butanol, and mixtures thereof.

9. The process according to claim 6, wherein the pharmaceutically acceptable excipient is selected from the group consisting of polyols, polymers, and mixtures thereof.

10. The process according to claim 9, wherein the polyol is selected from the group consisting of mannitol, maltitol, sorbitol, xylitol, erythritol, and isomalt, and mixtures thereof.

11. The process according to claim 9, wherein the polymer is selected from the group consisting of polyethylene glycol (PEG), copolymers of polyethylene glycol and polypropylene glycol, pentaerythritol, pentaerythritoltetraacetate, polymers of N-vinylpyrrolidone, polyoxyethylene stearates, poly-ε-caprolactone, hypromellose, microcrystalline cellulose, and mixtures thereof.

12. The process according to claim 6, wherein the removal of the solvent to isolate a premix of amorphous idelalisib is carried out by evaporation, solution concentration, filtration, spray drying, or lyophilization.

13. A process for the preparation of a premix of amorphous idelalisib, comprising the steps of:
a) dissolving idelalisib in a first solvent to form a first solution;
b) adding the solution to a second solvent to form a second solution;
c) adding a pharmaceutically acceptable excipient to the second solution; and
d) isolating a premix of amorphous idelalisib.

14. The process according to claim 13, wherein the dissolving step is carried out at an elevated temperature.

15. The process according to claim 13, wherein the first solvent is selected from the group consisting of N,N-dimethylformamide, N,N-dimethylacetamide, dimethyl sulfoxide, and mixtures thereof.

16. The process according to claim 13, wherein the second solvent is water.

17. The process according to claim 13, wherein the pharmaceutically acceptable excipient is selected from the group consisting of polyols, polymers, and mixtures thereof.

18. The process according to claim 17, wherein the polyol is selected from the group consisting of mannitol, maltitol, sorbitol, xylitol, erythritol, and isomalt, and mixtures thereof.

19. The process according to claim 18, where in the polyol is mannitol.

20. The process according to claim 17, wherein the polymer is selected from the group consisting polyethylene glycol (PEG), copolymers of polyethylene glycol and polypropylene glycol, pentaerythritol, pentaerythritoltetraacetate, polymers of N-vinylpyrrolidone, polyoxyethylene stearates, poly-ε-caprolactone, hypromellose, microcrystalline cellulose, and mixtures thereof.

21. The process according to claim 20, wherein the polymer is microcrystalline cellulose.

22. A premix comprising amorphous idelalisib and microcrystalline cellulose.

23. The premix of claim 22, comprising 10% w/w microcrystalline cellulose.

24. The premix of claim 22, comprising 30% w/w microcrystalline cellulose.

25. The premix of claim 22, comprising 50% w/w microcrystalline cellulose.

26. An oral dosage form comprising a premix of amorphous idelalisib and a pharmaceutically acceptable excipient.
